(19) United States
(12) Patent Application Publication
(10) Pub. No.: US 2008/0045932 A1
(43) Pub. Date: Feb. 21, 2008

(54) Unit Implantable Into a Living Body, Injection Medical System and Chronotherapeitic

(75) Inventors: Jacques Beau, Villabe (FR); Gerard Chevillon, Montrouge (FR); Francis Levi, Villejuif (FR)

Correspondence Address: YOUNG & THOMPSON, 745 SOUTH 23RD STREET, 2ND FLOOR, ARLINGTON, VA, 22202, US

(73) Assignee: B. Braun Medical Sas, Boulogne-Billancourt (FR)

(21) Appl. No.: 11/666,918

(22) PCT Filed: Nov. 3, 2005
(86) PCT No.: PCT/FR05/02733

§ 371 Date: Aug. 3, 2007

(30) Foreign Application Priority Data
Nov. 3, 2004 (FR) 0411730

(51) Int. Cl. A61K 9/22

(52) U.S. Cl. 604/891.1

(57) ABSTRACT

A therapeutic system, in particular to a medical injection system consisting of an external arrangement (12) includes a data receiver (20) and a data processing device (26) and of an injection arrangement (10) which is implantable into a living body and includes an injection device, a sensor of the living body parameter, a data transmitter (18) and a control and adjusting device (24) which determines a parameter measuring or data transmission condition such as a frequency of measurements for adapting the operation thereof to a patient actual state. The system is used for cancer and other disease chronotherapy.

The present invention concerns a unit implantable into a living body, a medical injection system intended for the optimisation of a therapy, and a method for therapeutic treatment of a human or animal patient, advantageously based on biological rhythms.

Although it applies to other treatments, intended for example for the therapy of infectious, metabolic, auto-immune, toxic, or traumatic diseases etc., the invention is described in its application to cancer therapy.

The majority of cancer patients receive chronic perfusions via an implanted venous access site. This site is particularly useful for ambulatory perfusions for continuous chemotherapy.

A chronotherapeutic approach to cancers has been proposed in order, on the one hand, to reduce the toxicity of the molecules and, on the other hand, to increase the efficacy thereof. Chemotherapy is thus carried out by chronomodulated perfusion with timetables determined according to a chronotherapeutic administration plan.

The therapy is based on the study of the circadian rhythm and its components (what are sometimes referred to as biological clocks). It has been demonstrated, for example, that non-steroidal anti-inflammatories were half as toxic for the stomach when they were taken in the evening rather than the morning. It was also demonstrated that the renal toxicity of gentamycin was lower when this antibiotic was taken through a perfusion in the middle of the day rather than at night.

The circadian rhythm comprises various components, including ultradian components. The latter have in general, for classic and non-pathological cases, a negligible influence. However, for particular cases, for example under the effect of administration of drugs, during the start of a pathological risk or under the action of a treatment or of an environmental event, these ultradian components are fundamentally informative and allow the taking of a decision and a possibility of early action.

The circadian rhythm is characterised by frequencies corresponding to periods of 24 hours and their harmonics: 12 hours, 8 hours, 6 hours, 4.8 hours, 4 hours, 3.43 hours, 3 hours, etc. The higher order harmonic components of this rhythm decrease in energy (and therefore in usefulness for defining the time structure). As a result, it does not appear useful to take temporal data for periods of less than 1 hour.

However, several factors may disturb the circadian system on which chronotherapy is based: individual variations; the pathology; and the therapy itself. Consequently, for a given patient, there may be a difference between the general time and that of the internal clock of the subject.

It has already been realised that the internal core temperature, which was regulated by the circadian system, constituted an excellent and strong marker of a patient's internal clock. Its measurement therefore gives a precise reference for the individual phase of the circadian rhythm and thus permits optimisation of the treatment by individualisation of the times of administration.

An endeavour has therefore already been made to record continuously the human body core temperature. However, no device is known which permits such recording except for in hospital, since only the measurement of the rectal temperature gives sufficiently convincing results, and this cannot be taken continuously in sick patients during extended periods.

It has thus been suggested to use a sensor associated with an implantable chamber intended for the execution of perfusions. The temperature sensor is intended to be implanted into the deep sub-cutaneous tissue, in contact with the muscles, and to be isolated from external thermal influences.

More precisely, consideration was given to associating with an implantable chamber a temperature sensor and a transmitter transmitting, at regular intervals, the value of the measured temperature to a receiver of an apparatus worn outside the body of the patient. The signals received by the receiver are intended to be recorded in the apparatus worn by the patient, then analysed by the medical staff, for whom the apparatus is intended.

The purpose of such a system is to know the phase of the circadian rhythm and its stability, for the definition of the treatment.

In addition, it was considered that the system could also permit early detection of the start of an infection or of ultradian rhythms. In fact, as indicated in FIG. 1, which is a graph showing the results of a study carried out by measurement of rectal temperatures, simple laryngitis causes a significant modification (curve shown by dashed lines) of the temperature which develops normally in the range contained between the two curves shown by solid lines (average over one week) with an amplitude generally of the order of 1±0.5° C. It is easy to imagine the interest of a warning given on detection of such an infection.

The aforesaid system has been designed, but not yet produced, to permit greater knowledge of the patient and better adaptation to the latter of the treatment. However, this adaptation is only global in so far as the results obtained correspond to general instructions, for example to a constant rhythm of transmission of the measured temperature, whatever the time of day and the state of the patient, so that the results obtained are only global.

It would be advantageous to obtain the results not only so that they may be used rapidly, and even very rapidly in the case of an accident, but also in a manner which is adapted to the state of the patient, especially to the variations which may occur, to the course of the pathology and to the consequences of the therapy.

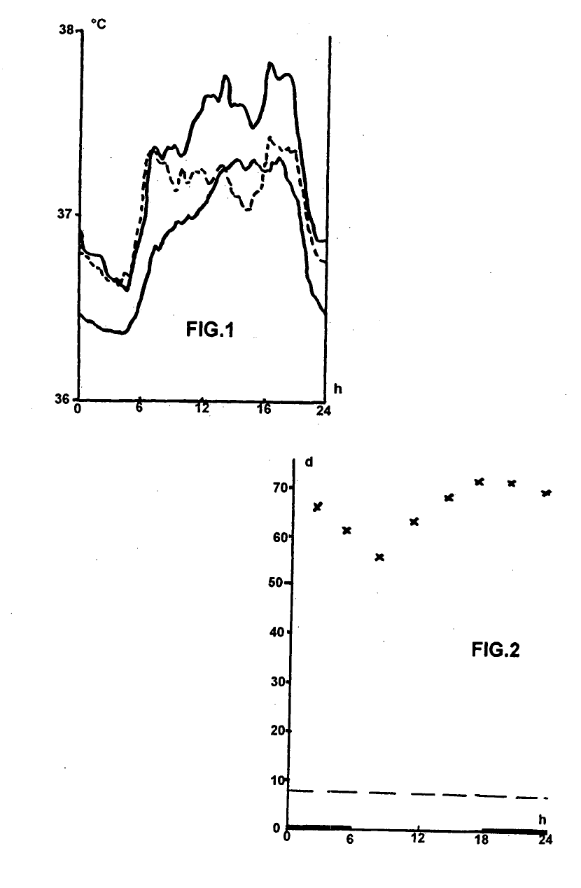

It is known in fact that refinement of the treatment of a cancer patient, for example by continuous perfusion or by repeated daily administration instead of only one, allows a considerable increase in the tolerance or the efficacy of the treatment. Moreover, tests carried out on mice showed that an injection every three hours of a quantity modulated sinusoidally over the day gives better results than an unmodulated injection, and especially than a single injection of a larger amount. Thus, FIG. 2 indicates that the survival time (in ordinates) of the mice reaches 55 to 72 days with modulation, while the single injection gives a survival time which is less than ten days.

The aim of the invention is to obtain precise and significant results obtained by measurement of at least one parameter of a living body, preferably the core temperature of the living body, at a frequency adapted to the state of the body, with a system which is as simple and robust as possible, utilising already proven technologies for the exchange of data between a device implanted into a living body and an external device.

Numerous systems are already known in which a unit implantable into a living body comprises a sensor for at least one parameter, and a data transmitter associated with an antenna and which carries out the transmission of data representing the parameter at a frequency of transmission, the data being intended for an external receiver.

Thus, the document U.S. Pat. No. 4,871,351 describes a system which comprises an implantable assembly having a drug administering pump controlled by data exchange between the implantable assembly and an external assembly. This system transmits data relating to the functioning of the implantable assembly, but does not suggest the regular measurement of a parameter of the living body into which the assembly is implanted.

Similarly, the documents FR-2 792 841, US2002/042596, US2002/156462 and EP-672 427 describe systems including an implantable unit, sometimes performing a measurement, but always of an operating parameter of the implanted apparatus and not of the living body. Moreover, none of these suggests the modulation of the frequency of measurement depending on the results of the measurements. Only placing on standby is suggested, for energy saving reasons, and not depending on the results.

The aim of the invention is to take advantage of the known technology of data communication between an implantable assembly and an external assembly in order to refine the knowledge of the current state of the patient, and therefore to increase the efficacy of the treatment of the patient or the surveillance thereof, by adapting the measuring conditions either directly, or with local data processing, by means of the modulation of the measuring conditions.

More precisely, the invention concerns a unit implantable into a living body, of the type which comprises a sensor for at least one parameter of the living body, and a data transmitter associated with an antenna and which carries out the transmission of data representing the parameter at least at a frequency of transmission, the data being intended for an external receiver; according to the invention, the sensor includes a pick-up for at least one signal representing at least one living body parameter, and a sampling device connected to the pick-up is intended to sample the value of the signal from the pick-up at a frequency of measurement, and the implantable unit includes a control and adjustment device intended to adjust the frequency of measurement.

Preferably, the control and adjustment device also adjusts the frequency of transmission of the data depending on the frequency of measurement.

In an advantageous embodiment, the implantable unit further includes a memory containing data representing at least one cycle of variations of the data, the cycle of variations advantageously having a duration of twenty-four hours.

In an advantageous embodiment, the control and adjustment device further includes a warning data creation device intended to transmit data. For example, the warning data are obtained by comparison of values of the signal from the pick-up with an upper or lower threshold, or by comparison of sampled values with an upper or lower threshold.

Preferably, the warning data are intended to trigger an operation of processing of the values sampled by the control and adjustment device, for example depending on the value of the pick-up signal or the sampled value, depending on the variation of the value of the pick-up signal or the sampled value, depending on the difference between consecutive values of the pick-up signal or the sampled value, or depending on the result of the comparison of the value of the pick-up signal or the sampled value with a stored value, the stored value having been deduced, for example, from the data picked up 24 hours previously or a multiple of 24 hours previously.

Preferably, the pick-up is a pick-up for the core temperature of the living body, although other parameters may be measured, such as a parameter representing the rest/activity state, the concentration of glucose or of the calcium ion, other parameters linked to the arterial pressure or to the cardiac rhythm and other parameters of a physical, chemical or biological nature, such as the pH of a fluid. A plurality of sensors may advantageously measure a plurality of parameters.

Preferably, the sampling device and the control and adjustment device, advantageously with the warning device, form a single electronic device.

In an advantageous embodiment, the invention permits bidirectional interaction between the implanted part and the external part. In fact, it is scarcely possible to envisage implanting an electronic system capable of performing, by itself, significant data processing taking into account numerous parameters, since it would have to be extremely elaborate and bulky, and have a high power consumption. Consequently, in a base system, only the conditions of measurement and transmission of a parameter may be modified by the external system. In a more elaborate system, the implanted device itself executes summary data processing, for example permitting the modulation of the frequency of measurement and/or the determination of a warning condition.

The invention thus concerns a medical injection system intended for the optimisation of a therapy which comprises an external assembly, including a first data receiver, and an injection assembly implantable into a living body, and comprising on the one hand an injection device and on the other hand an implantable unit such as defined in the preceding paragraphs.

Preferably, the external assembly comprises a second data transmitter and a data processing device, and the implantable injection assembly comprises a second data receiver intended to receive instruction data from the second transmitter, and a control and adjustment device intended, depending on the instructions received by the second receiver, to determine at least one condition selected from the conditions for measurement of the living body parameter by the sensor and the conditions for transmission, by the first transmitter, of the data corresponding to a value measured by the sensor.

In one embodiment, the external assembly comprises at least two parts, a first: part including at least one recording device for data coming from the implantable assembly, and a second part including the data processing device, the second part being intended to receive, on command, data from the data recording device.

Preferably, the processing device of the external assembly is intended to adapt the instruction data on the one hand to the data received from the first transmitter and on the other hand to other data relating to the living body into which the implantable assembly is implanted.

In one embodiment, a condition determined by the control and adjustment device comprises a condition at least linked to the frequency with which the sensor measures the living body parameter.

In an exemplary embodiment, a condition determined by the control and adjustment device comprises a condition at least linked to the specific period of execution of the transmission of the data relating to the living body parameter by the first transmitter.

Preferably, the control and adjustment device of the implantable assembly is additionally intended to carry out data processing.

Preferably, the data processing carried out by the control and adjustment device comprises a process of compression of the data to be transmitted by the first transmitter.

In an advantageous embodiment, the system further includes a warning device, and the control and adjustment device of the implantable assembly triggers, directly or otherwise, the warning device when a condition linked to the parameter measured by the sensor is fulfilled. For example, the warning device is held by the external part worn by the patient who can immediately be advised, for example by means of a detailed message displayed on that part. In one example, the condition linked to the parameter measured is the exceeding of at least one lower or upper limit value of the parameter.

Preferably, the implantable injection assembly is a chamber implantable into the deep subcutaneous tissue, intended for the diffusion or collection of liquid, or an implantable liquid injection pump.

Preferably, the implantable injection assembly further includes an independent electrical supply.

The invention also concerns a method for the treatment of a disease of a human or animal patient, of the type which comprises the determination of the circadian rhythm of the patient affected by the disease by measurement of the patient's core temperature, and the administration to the patient of at least one composition effective for the treatment of the patient's disease at at least one moment in the day determined according to the circadian rhythm; according to the invention, the step of determining the patient's circadian rhythm comprises the measurement of the patient's core temperature at a frequency of measurement determined by an implanted unit, the evaluation of the state of the patient by comparison of the circadian rhythm thus determined continuously with a reference circadian rhythm of the patient in the implanted unit, and, according to the state thus determined, the execution of at least one operation selected from the modification of the frequency of measurement, the transmission of specific warning data, and the modification of the administration of an effective constituent.

Preferably, the method comprises obtaining specific warning data by comparison of values of the measured signal or sampled values with an upper or lower threshold.

Preferably, the step of execution of at least one operation comprises the execution of an operation of processing of values sampled by the control and adjustment device depending on the value of the pick-up signal or the sampled value, on the variation of the value of the measured signal or of the sampled value, on the difference between consecutive values of the measured signal or of the sampled value, or on the result of the comparison of the value of the measured signal or of the sampled value with a stored value.

The method is intended, for example, for the treatment not only of cancers, but also for that of infectious, metabolic, auto-immune, toxic, or traumatic diseases, etc.

Other characteristics and advantages of the invention will become clearer on reading the following description of exemplary embodiments, provided with reference to the appended drawings, in which:

FIGS. 1 and 2 have already been described;

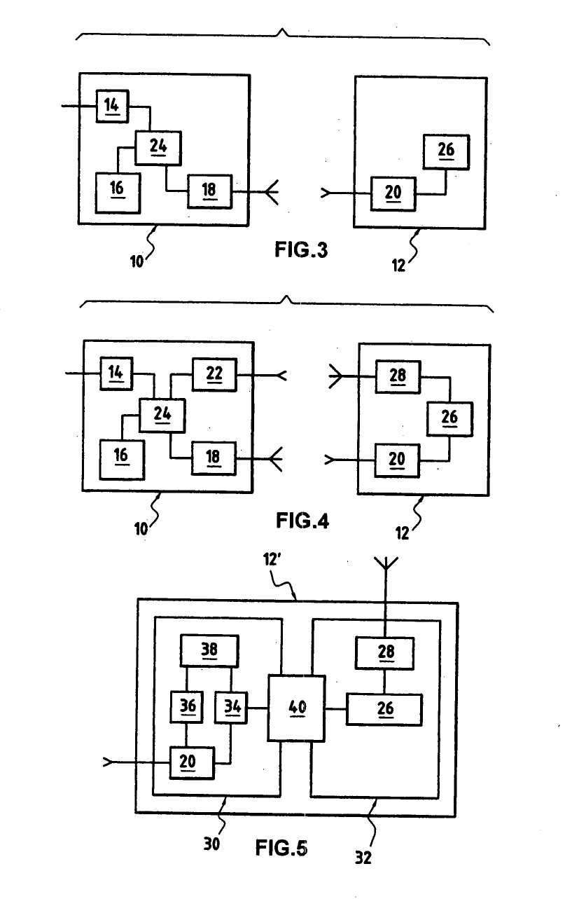

FIG. 3 is a synoptic diagram representing the principal elements of a medical device for the optimisation of a therapy according to the invention;

FIG. 4 is a synoptic diagram representing the principal elements of a bidirectional medical device for the optimisation of a therapy according to the invention; and

FIG. 5 shows a variant of the external part of the device of FIG. 4.

FIG. 3 shows a medical device which includes an implantable assembly 10 and an external assembly 12.

In this unidirectional type system, the implantable assembly 10 is an implantable chamber equipped with an electronic module comprising a temperature sensor 14, comprising a pick-up and a sampling device, a power supply 16 (generally an electrical battery), and a transmitter 18 which is intended to transmit data corresponding to the value of the temperature obtained by the sensor 14. A control and adjustment device 24 ensures the functioning of the whole of the system. The data are received by a receiver 20 of an external assembly 12 which also includes a processing circuit 26, are recorded, and then used for the evaluation of the patient's circadian rhythms and of their course.

FIG. 4 shows a bidirectional type system which also includes an implantable assembly 10 and an external assembly 12. In addition to the elements described with reference to FIG. 3, the implantable assembly 10 includes a receiver 22, and the external assembly includes a second data transmitter 28 capable of transmitting signals intended for the receiver 22.

The signals received by the receiver 22 are processed by the control and adjustment circuit 24 so that the latter carries out or controls various operations: variation of the sensing rhythm for the parameter or parameters, variation of the rhythm of transmission of the data by the transmitter 18, determination of urgent conditions requiring the triggering of a warning, etc.

In an exemplary embodiment, the implantable assembly 10 is formed by a double implantable chamber equipped with reservoirs of reduced dimensions so that the electronic circuit and the antenna can be housed. The total volume of the electronic circuit of the implantable assembly is less than 1 cm³. When the parameter measured is the temperature, the sensor gives a resolution of 0.1° C. and an absolute accuracy of 0.2° C., in the range of temperatures between 35 and 42° C. The sampling of the temperature may be carried out every minute, and even more frequently in case of urgency, or every ten minutes or even every hour or more in a rest period.

The antenna of the implantable chamber may occupy only a small volume. Thus, it may be constituted by a simple loop arranged, for example, at one side of the chamber, when the reservoirs are metallic. As this arrangement may present transmission constraints, especially of directivity of the transmitted signals, it is possible to use other embodiments.

In a first variant, the reservoir or reservoirs are not conductive: they are formed for example from ceramics, so that an antenna loop may surround practically all of the chamber. In a second variant, at least one reservoir, circular in shape, may itself constitute an antenna, for example by deposition of an antenna design on a reservoir made of a non-conductive material. In a third variant, a long effective antenna may be arranged along the catheter to which the implantable chamber is connected.

FIG. 5 shows a variant of the external assembly 12. The external assembly 12′ comprises two separable parts 30 and 32. The part 30 is intended to be transported by the patient, while the part 32 is intended to be located in medical premises.

The part 30 transportable by the patient comprises not only the data receiver 20, but also a data recording device 34, a control device 36 and, preferably, a warning device 38. The part 32 intended to be located in medical premises comprises the data processing circuit 26 and optionally the second transmitter 28, although this latter may also be situated in the transportable part 30.

The reference 40 designates a device for temporary connection of the part 30 transportable by the patient and of the part 32 intended to be located in medical premises. The connection device is bidirectional. It may consist of electrical connectors. However, it may also be a wireless connection, produced between transmitter/receivers arranged one in the transportable part 30 and the other in the part 32 intended to be located in medical premises.

The reference 38 designates a warning device, for example a buzzer, a vibrator or a high frequency link to a management centre. This device is preferably incorporated in the part transportable by the patient. In this way, when the control and adjustment circuit 24 of the implantable part 10 detects an alert condition, for example excessively rapid or abnormal variation of the course of at least one measured parameter, in comparison with value or variation thresholds, or quite simply the lack of functioning of any element, it commands the transmission of a warning signal via the transmitter 18, although the control circuit 36 of the transportable part 30 may determine this alert condition and order the warning 38. The user is preferably advised of the nature of the alert via a message presented on a display.

Naturally, for security reasons, the control and adjustment circuit 24 may command, in a known manner, the transmission of a signal indicating that the implantable device is functioning effectively, so that, in the absence of reception of any signal, the control circuit 36 of the transportable part may determine this absence of transmission or the disturbance of the transmission and deduce therefrom the existence of a transmission fault. This system will not be described in more detail, since it is well known in the field of safety devices.

The presence of a control and adjustment circuit 24 permits summary processing of the data from the sensor 14, intended to reduce the number of operations which consume the most power. In fact, the power supply 16 of the implantable part should be able to ensure functioning for several years, and it is therefore necessary not only that it is of small volume and therefore a high power density, but also that the power consumed is as low as possible.

One of the essential functions of the control and adjustment circuit 24 is to reduce as much as possible the operation of the transmitter, either by reduction of its duration or its frequency of operation, or by reduction of the data transmitted. For example, the transmission of a signal indicating that the parameter maintains the same value as the preceding signal consumes less power than the transmission of a signal representing the value itself. Such processing should obviously be carried out in the implantable part.

In an example of determination of the parameter controlled which is the transmission rhythm, the transmission by the implantable part has a long periodicity if the parameter does not vary significantly, and shorter in the contrary case, optionally with intermediate “beeps” indicating that the system has not broken down.

The modulation of the measuring conditions, however, presents certain problems. According to the Nyquist theorem, sampling at a certain period makes it possible to access double period data (corresponding to the Nyquist frequency). If components of the sampled signal have a significant energy beyond the Nyquist frequency (twice the sampling frequency), the picking up of the sampled signal is fundamentally erroneous, and a stroboscopic effect may for example be observed.

It is therefore necessary that the sampling frequency is sufficient when the rhythm of development of the measured parameter (the temperature in the case under consideration) is disturbed rapidly, and that the speed of sample taking increases. In practice, if sampling is carried out at a sufficiently high frequency, the signal is suitably sampled, and it is possible to deduce therefrom that this frequency is too high and therefore reduce the number of useful samples to be recorded or to be transmitted. If the signal is sampled at an insufficient frequency, it is not possible to know whether it is necessary to increase that frequency, since the signal is wrongly sampled.

More elaborate analyses may also be carried out such as, for example, calculation of the spectrum by Fourier transform or filtering steps adapted in order to determine the optimum period for conservation of data.

To summarise, if sampling is carried out too quickly in relation to a frequency judged to be optimum, the signal is suitably picked up, but the quantity of information retained and the power expended are excessive; if sampling is not carried out quickly enough in relation to a frequency judged to be optimum, the signal is not picked up suitably and cannot be recovered. The frequency judged to be optimum varies, however, with the aggression undergone by the patient, the regularity or irregularity of the disturbance which the aggression causes, etc., such that the modulation of the measuring conditions depends on the patient himself.

Thus, the modulation of the measuring conditions, and therefore the data processing carried out at least in part in the control and adjustment circuit 24, has a paramount effect on the quality of the results obtained and on the power resources necessary in the implanted assembly.

A system has been considered in which the warning device is incorporated in the transportable part of the external assembly. However, it is also possible to execute the warning function in the part 32 located in the medical premises. In this case, it is advantageous for the connection 40 between the parts 30 and 32 of the external assembly to be wireless, for example by means of a portable telephone or another high frequency connection.

In fact, the processing device 26 of the external assembly 32 may be a common processing device which manages data relating to numerous patients and which permits the determination of conditions which could not be determined from a single patient.

Such a type of connection permits the adaptation of the medical treatment of each patient not only to the individual data of the patient, but also to other data concerning, for example, groups of patients, and makes it possible to enable each patient to benefit as soon as possible from the knowledge obtained of the course of the disease from a plurality of patients or from a group of patients simultaneously.

Thus, the decisional prescriptions of the medical personnel may take into account knowledge acquired from a whole group of patients.

To summarise, the invention concerns a medical system which makes it possible to know intrinsic and developmental characteristics of the circadian rhythm of the patient concerned, and to adapt the treatment according to those characteristics.

The invention also concerns the application of this medical system to therapeutic treatment of diseases, especially cancers.

Naturally, various modifications may be applied by an expert in the field to the systems which have been described solely by way of non-limiting example without departing from the scope of the invention.

1. A unit implantable into a living body, of the type which comprises:
a sensor (14) for at least one parameter of the living body, and
a data transmitter (18), associated with an antenna and which carries out the transmission of data representing the parameter at least at a frequency of transmission, the data being intended for an external receiver,
characterized in that
the sensor (14) includes
a pick-up for at least one signal representing at least one parameter of the living body, and
a sampling device connected to the pick-up and intended to sample the value of the pick-up signal at a frequency of measurement, and
the implantable unit (10) includes a control and adjustment device (24) intended to adjust the frequency of measurement.

2. A unit according to claim 1, characterized in that the control and adjustment device (24) also adjusts the frequency of data transmission depending on the frequency of measurement.

3. A unit according to claim 1, characterized in that the implantable unit (10) further includes a memory containing data representing at least one cycle of variations of the data.

4. A unit according to, characterized in that the control and adjustment device (24) further includes a warning data creation device intended to transmit data.

5. A unit according to claim 1, characterized in that the pick-up is a pick-up for the core temperature of the living body.

6. A unit according to claim 4, characterized in that the sampling device and the control and adjustment device (24) form a single electronic device, in co-operation with the warning device.

7. A medical injection system intended for the optimisation of a therapy, characterized in that it comprises:
an external assembly (12), comprising a first data receiver (20), and
an injection assembly (10) implantable into a living body, and comprising on the one hand an injection device and on the other hand an implantable unit (10) according to claim 1.

8. A system according to claim 7, characterized in that the external assembly (12, 12′) comprises a second data transmitter (28) and a data processing device (26), and the implantable injection assembly (10) comprises a second data receiver (22) intended to receive instruction data from the second transmitter (28), and a control and adjustment device (24) intended, depending on the instructions received by the second receiver (22), to determine at least one condition selected from the conditions for measurement of the living body parameter by the sensor (14) and the conditions for transmission, by the first transmitter (18), of the data corresponding to a value measured by the sensor (14).

9. A system according to claim 7, characterized in that the external assembly (12′) comprises at least two parts, a first part (30) including at least one device (34) for recording data coming from the implantable assembly, and a second part (32) including the data processing device (26), the second part (32) being intended to receive, on command, data from the data recording device (34).

10. system according to claim 8, characterized in that the processing device (26) of the external assembly (32) is intended to adapt the instruction data on the one hand to the data received from the first transmitter (18) and on the other hand to other data relating to the living body into which the implantable assembly (10) is implanted.

11. A system according to claim 8, characterized in that a condition determined by the control and adjustment device (24) comprises a condition at least linked to the frequency with which the sensor (14) measures the living body parameter.

12. A system according to claim 8, characterized in that a condition determined by the control and adjustment device (24) comprises a condition at least linked to the specific period of execution of the transmission of data relating to a living body parameter by the first transmitter (18).

13. A system according to claim 8, characterized in that the control and adjustment device (24) of the implantable assembly is further intended to carry out data processing.

14. A system according to claim 13, characterized in that the data processing carried out by the control and adjustment device (24) of the implantable assembly comprises a process of compression of the data to be transmitted by the first transmitter (18).

15. A system according to claim 8, characterized in that it further includes a warning device (38), and in that the control and adjustment device (24) of the implantable assembly triggers, directly or otherwise, the warning device (38) when a condition linked to the parameter measured by the sensor (14) is fulfilled.

16. A system according to claim 15, characterized in that the condition linked to the parameter measured is that of exceeding at least one lower or upper limit value of the parameter.

17. A method for the treatment of a disease of a human or animal patient, of the type which comprises:
the simultaneous determination of circadian and ultradian rhythms of the patient affected by the disease by measurement of the patient's core temperature, and
the administration to the patient of at least one composition effective for the treatment of the patient's disease at least one moment of the day determined according to the circadian and ultradian rhythms,
characterized in that the step of determination of the patient's circadian and ultradian rhythms comprises:
the measurement of the patient's core temperature at a frequency of measurement determined by an implanted unit (10),
the evaluation of the state of the patient by comparison of the circadian rhythm thus determined continuously with the patient's reference circadian rhythm in the implanted unit (10), and
according to the state thus determined, the execution of at least one operation selected from the modification of the frequency of measurement; the transmission of specific warning data; and the modification of the administration of an effective constituent.

18. A method according to claim 17, characterized in that it comprises obtaining specific warning data by comparison of values of the measured signal or of sampled values with an upper or lower threshold.

19. A method according to either claim 17, characterized in that the execution of at least one operation comprises the execution of an operation of processing values sampled by the control and adjustment device (24) depending on the value of the pick-up signal or the sampled value, on the variation of the value of the measured signal or the sampled value, on the difference between consecutive values of the measured signal or of the sampled value, or on the result of the comparison of the value of the measured signal or the sampled value with a stored value.

20. A method according to either claim 18, characterized in that the execution of at least one operation comprises the execution of an operation of processing values sampled by the control and adjustment device (24) depending on the value of the pick-up signal or the sampled value, on the variation of the value of the measured signal or the sampled value, on the difference between consecutive values of the measured signal or of the sampled value, or on the result of the comparison of the value of the measured signal or the sampled value with a stored value.
